(19) United States
(12) Patent Application Publication
(10) Pub. No.: US 2010/0151025 A1
(43) Pub. Date: Jun. 17, 2010

(54) PREVENTION AND TREATMENT FOR OSTEONECROSIS AND OSTEORADIONECROSIS OF THE JAW

(75) Inventors: Samuel E. Lynch, Franklin, TN (US); Jeffrey O. Hollinger, Gibsonia, PA (US); Leslie A. Wisner-Lynch, Franklin, TN (US)

Correspondence Address: Biomimetic Therapeutics Inc.;C/O MoFo Palo Alto, 755 Page Mill Road, Palo Alto, CA, 94304-1018, US

(73) Assignee: BIOMIMETIC THERAPEUTICS, INC., Franklin, TN (US)

(21) Appl. No.: 12/527,692

(22) PCT Filed: Feb. 20, 2008
(86) PCT No.: PCT/US2008/054354

§ 371 Date: Feb. 24, 2010

Related U.S. Application Data
(60) Provisional application No. 60/890,763, filed on Feb. 20, 2007.

(51) Int. Cl. A61K 9/10; A61K 38/18; A61P 19/08

(52) U.S. Cl. 424/484; 514/12

(57) ABSTRACT

The present invention provides compositions and methods useful for treating, preventing or slowing the progression of ONJ and ORNJ. The invention provides for the use of PDGF in a pharmaceutically acceptable buffer in the preparation of a medicament useful for treating, preventing or slowing the progression of ONJ and ORNJ. The invention provides for the use of PDGF in a pharmaceutically acceptable buffer wherein the PDGF is disposed in a biocompatible matrix in the preparation of a medicament useful for treating, preventing or slowing the progression of ONJ and ORNJ. In one embodiment, a method for treating, preventing or slowing the progression of ONJ or ORNJ comprises providing a composition comprising a PDGF solution disposed in a biocompatible matrix and applying the composition to a desired site in the jaw. In another embodiment, a method for treating, preventing or slowing the progression of ONJ or ORNJ comprises providing a composition comprising a PDGF in a pharmaceutically acceptable buffer and applying the composition to a desired site in the jaw. The present invention also provides kits useful for treating, preventing or slowing the progression of ONJ and ORNJ.

### FIELD OF THE INVENTION

The present invention relates to compositions, methods and kits useful for treating, preventing or slowing the progression of osteonecrosis of the jaw and osteoradionecrosis of the jaw.

### BACKGROUND OF THE INVENTION

Osteonecrosis of the jaw (ONJ) can be a pathological sequel to dental surgical procedures in certain patients. Although a definitive ONJ etiology has not been determined, there is a growing concern that the most susceptible patients who may develop ONJ are receiving bisphosphonates and have co-morbidities of metastatic bone cancer (e.g., from prostate, breast, lung, kidney), multiple myeloma, osteogenesis imperfecta and Paget's disease.

The etiology of ONJ may be related to decreased angiogenesis. Angiogenesis is a pivotal process antecedent to mucosal healing, periodontal regeneration and osteogenesis. It has been reported that platelet-derived growth factor (PDGF) and vascular endothelial growth factor (VEGF) are decreased in patients on bisphosphonates. PDGF induces new blood vessel formation and VEGF promotes endothelial cell maturation of the endothelial cells during angiogenesis. Consequently, the tandem effect of the vulnerary factors PDGF and VEGF is compelling for new blood vessel formation and the maturation and functional stability of blood vessels at the wound healing site.

When blood vessels do not develop, tissue wound healing does not occur and mucosal tissue precipitously degenerates, as seen in osteoradionecrosis (ORNJ) of the jaw in cancer patients who have had radiation therapy. The absence or decrease in blood vessels, poor soft tissue wound healing and mucosal breakdown and dehiscence are clinical signs common to ONJ and ORNJ.

Moreover, vascular deficiency at a healing bone wound has the additional ramification of decreasing pre-osteoclast lineage cells, specifically, blood born monocytes that may become pre-osteoclasts. The significance of a decrease in the pre-osteoclast progenitor population is highly relevant to the remodeling of both healing bone and bone homeostasis. It has been suggested that the high turnover (i.e., remodeling) in the mandible and maxilla requires a balance between bone forming cells, osteoblasts, and bone resorbing cells, osteoclasts. Bisphosphonates disrupt osteoclastic bone resorption, decrease osteoclast function and increase osteoclast apoptosis (i.e., cell death). Furthermore, the decrease in vasculature in the mandible and maxilla as a consequence of bisphosphonates, limits osteoclast cell renewal by significantly limiting monocyte transit through blood vessels and their subsequent lineage progression to osteoclasts. As a consequence of bone cell imbalance (i.e., the osteoclast:osteoblast ratio), remodeling becomes uncoupled and there is a loss of bone homeostasis (i.e., bone metabolism: replacement and renewal of damaged bone). Therefore, microfractures in the mandible and maxilla are not adequately repaired, pre-disposing this region to ONJ. Furthermore, the mandible and maxilla are high bone turnover regions due to continuous biomechanical stimuli from mastication and swallowing, and bisphosphonate-induced homeostatic imbalance predisposes the oral region to ONJ.

It is known that bisphosphonates inhibit osteoclast formation and activity, as well as viability. Bisphosphonates are incorporated into calcified tissues, for example, bone, and may have a half life in bone for up to 12 years. Thus, bone resorbing osteoclasts that internalize bone fragments during resorption, will be significantly affected. It is noteworthy to mention that bone turnover, the homeostatic process of remodeling that involves the osteoblast-osteoclast coupling, results in complete skeletal remodeling of bone every 10 years. Consequently, the bisphosphonate effects on osteoclasts will have a significant effect on homeostatic bone turnover and the magnitude of that effect will be determined by the chemistry of the bisphosphonates.

Bisphosphonates may be categorized as either containing amine groups or non-amine groups. The aminobisphosphonates are the contemporary class of the drug and affect osteoclasts by inducing adenosine triphosphate analogues that are cell cytotoxic, thereby prompting apoptosis as well as inhibiting farnesyl diphosphate synthase. The outcome disrupts osteoclast intracellular transport and causes cytoskeletal disorganization. Moreover, it is hypothesized that the aminobisphosphonates promote upregulation of osteoclast inhibitory factors from osteoblasts.

The overall therapeutic benefits of bisphosphonates are profound and compelling for osteoporotic patients and especially for patients with multiple myeloma, osteogenesis imperfecta, Paget's disease, and cancer metastasis. However, the disadvantages of prolonged bisphosphonate therapy for osteoporotics are prompting a strategic reevaluation of the bisphosphonate regimens. For example, as a consequence of the uncoupling action between osteoclasts and osteoblasts caused by bisphosphonates, remodeling is negatively impacted. Consequently, despite the deposition of new bone that may appear to be mineral dense, the new bone is actually biomechanically incompetent and therefore susceptible to fracture. The bone deposited is in fact osteopetrotic-like. Therefore, a strategy that allows ‘an escape’ from bisphosphonates is being promulgated by many in the medical community.

ORNJ is caused by radiation. Jaw tumors can be treated with radiation to eradicate the tumor. However, the ramifications of radiation treatment can result in localized tissue hypoxia, hypocellularity and hypovascularity. These sequelae are effective in eradicating oncological activity, which for solid state tumors involves hypercellularity and hypervascularity.

ORNJ can be reversed by increasing local vascularity. Hyperbaric oxygen (HBO) treatment is an effective treatment for ORNJ. HBO appears to increase the formation of new blood vessels. However, HBO has not been effective for ONJ. The reason for the lack of benefit with HBO for ONJ is that ONJ has a different etiology than ORNJ. The specific delineating and distinguishing difference between etiologies of ORNJ and ONJ is that the latter pathology, in addition to being hypovascular, is also an osteoclast-osteoblast uncoupling, resulting in a remodeling imbalance. This imbalance appears to increase the local susceptibility of healing bone to break down, thereby becoming necrotic. Therefore, while there is an overlapping similarity in etiology due to the vascularity issue (i.e., hypovascularity), ONJ has the additional etiologic involvement of a remodeling imbalance between osteoblasts and osteoclasts that does not appear to be consistent with ORNJ.

Currently there are no products available to prevent ONJ. Existing treatments for ONJ are palliative and consist of various protocols that may include debridement of necrotic bone with very limited extension to viable bleeding bone, an analgesic, chlorhexidine rinses and antibiotics.

In view of the foregoing problems, it would be desirable to provide compositions and methods for treating ONJ. It would also be desirable to provide compositions and methods for preventing ONJ or delaying its progression in patients at risk for developing ONJ. Also needed are compositions and methods for preventing and treating ORNJ or delaying its progression.

### SUMMARY

The present invention addresses these needs by providing compositions and methods useful for treating ONJ, for preventing ONJ or delaying its progression in patients at risk for developing ONJ, and for treating and/or preventing ORNJ or delaying its progression.

The present invention provides for the use of PDGF in a pharmaceutically acceptable vehicle in the preparation of a medicament for treating, preventing, or slowing the progression of ONJ or ORNJ in a patient. The present invention provides for the use of composition comprising a solution of PDGF in a pharmaceutically acceptable vehicle disposed in a biocompatible matrix in the preparation of a medicament for treating, preventing, or slowing the progression of ONJ or ORNJ in a patient.

In one embodiment, these compositions comprise PDGF in a biocompatible matrix. In another embodiment, the composition comprises a solution of PDGF in a pharmaceutically acceptable buffer. Individuals receiving bisphosphonates (orally or intravenously) with or without co-morbidities of metastatic bone cancer, multiple myeloma and Paget's disease are susceptible to ONJ and will significantly benefit from a prophylactic treatment to prevent ONJ. Prophylactic treatment is appropriate for a patient population at risk for ONJ.

Individuals receiving radiation treatment, for example during cancer therapy are susceptible to ORNJ and will significantly benefit from a treatment for ORNJ. Prophylactic treatment is appropriate for a patient population at risk for ORNJ.

Prevention emphasizes prophylaxis, which means co-administration of the PDGF-containing composition concurrently with the dental procedure. For example, a patient at risk and having a dental surgical procedure such as an extraction would have the PDGF-containing composition, in one embodiment, co-administered with, for example, a dental extraction medicament or dressing. Yet another example for use of the PDGF-containing composition is an oro-dental cystrectomy where the PDGF-containing composition is placed into the cystic cavity. Still another example includes a periodontal procedure where gingival tissues were incised and alveolar and/or inter-radicular osseo-dental surgery were performed and the PDGF-containing composition is co-administered with the periodontal therapy dressing.

In some embodiments, the quantity of the PDGF-containing composition administered is determined by the bone volume that had been surgically removed, for example from an extraction socket, a cystrectomy, or during periodontal bone surgery.

The administration of a PDGF-containing therapeutic composition administered for bone healing procedures is compelling. While not wanting to be bound by the following statement, it is believed that PDGF, as a chemoattractant, will recruit mesenchymal cells, including but not limited to osteoblasts, osteoclasts, mesenchymal stem cells, fibroblasts and vascular smooth muscle cells, to a healing bone wound that may be cell-poor. Further, the mitogenic property of PDGF will amplify or increase the quantity of the recruited cells to the bone healing site by inducing mitogenesis (i.e., cell replication). The consequence is that this enriched cell pool can become a healing blastema for patients who may otherwise have had long term bisphosphonate treatment that significantly reduced the cell population and activity of that population necessary for bone healing. While not wanting to be bound by the following statement, it is also believed that the angiogenic properties of PDGF can help to restore vascularity to the hypovascular site of ORNJ.

In some embodiments of the present invention, there are provided compositions and methods for treating or delaying the progression of ONJ. In some embodiments of the present invention, there are provided compositions and methods for preventing ONJ.

In some embodiments of the present invention, there are provided compositions and methods for treating or delaying the progression of ORNJ. In some embodiments of the present invention, there are provided compositions and methods for preventing ORNJ.

In one aspect, the present invention provides a composition for treating, preventing or delaying the progression of ONJ comprising a solution comprising PDGF and a biocompatible matrix, wherein the solution is disposed in the biocompatible matrix. This composition is applied to a desired site, such as a site of osteonecrosis or a site vulnerable to osteonecrosis.

In another aspect, the present invention provides a composition for treating, preventing or delaying the progression of ORNJ comprising a solution comprising PDGF and a biocompatible matrix, wherein the solution is disposed in the biocompatible matrix. This composition is applied to a desired site, such as a site of osteonecrosis or a site vulnerable to osteonecrosis.

In some embodiments, PDGF is present in the solution in a concentration ranging from about 0.01 mg/ml to about 10 mg/ml, from about 0.05 mg/ml to about 5 mg/ml, or from about 0.1 mg/ml to about 1.0 mg/ml. The concentration of PDGF within the solution may be within any of the concentration ranges stated above.

In some embodiments of the present invention, PDGF comprises PDGF homodimers and heterodimers, including PDGF-AA, PDGF-BB, PDGF-AB, PDGF-CC, PDGF-DD, and mixtures and derivatives thereof. In one embodiment, PDGF comprises PDGF-BB. In another embodiment PDGF comprises a recombinant human (rh) PDGF such as recombinant human PDGF-BB (rhPDGF-BB). In some embodiments, PDGF comprises mixtures of the various homodimers and/or heterodimers. Embodiments of the present invention contemplate any combination of PDGF-AA, PDGF-BB, PDGF-AB, PDGF-CC and/or PDGF-DD.

In some embodiments of the present invention, PDGF comprises one or more PDGF fragments. In one embodiment rhPDGF-B comprises the following fragments: amino acid sequences 1-31, 1-32, 33-108, 33-109, and/or 1-108 of the entire B chain. The complete amino acid sequence (1-109) of the B chain of PDGF is provided in FIG. 15 of U.S. Pat. No. 5,516,896. It is to be understood that the rhPDGF compositions of the present invention may comprise a combination of intact rhPDGF-B (1-109) and fragments thereof. Other fragments of PDGF may be employed such as those disclosed in U.S. Pat. No. 5,516,896. In accordance with one embodiment, the rhPDGF-BB comprises at least 60% of the entire amino acid sequence of intact rhPDGF-B (1-109).

A biocompatible matrix, according to some embodiments of the present invention, comprises a scaffolding material. In some embodiments, a scaffolding material comprises calcium phosphate. Calcium phosphate, in one embodiment, comprises β-tricalcium phosphate (β-TCP).

In another aspect, the present invention provides a composition useful to treat, prevent or delay the progression of ONJ or ORNJ, comprising a PDGF solution disposed in a biocompatible matrix, wherein the biocompatible matrix comprises a scaffolding material and a biocompatible binder. In some embodiments, a biocompatible binder comprises a material operable to promote adhesion between combined substances. A biocompatible binder, for example, can promote adhesion between particles of a scaffolding material in the formation of a biocompatible matrix. In one embodiment, a biocompatible binder comprising collagen can promote adhesion between β-TCP particles of a scaffolding material. Moreover, the PDGF solution disposed in a biocompatible matrix comprising a scaffolding material and a biocompatible binder may have a concentration of PDGF as described herein.

A biocompatible binder, according to some embodiments of the present invention, comprises proteins, polysaccharides, glycosaminoglycans, nucleic acids, carbohydrates, synthetic polymers, or mixtures thereof. In one embodiment, a biocompatible binder comprises collagen. In another embodiment, a biocompatible binder comprises hyaluronic acid. In a further embodiment, a biocompatible binder comprises chitosan or elastin. Moreover, biocompatible matrices, including biocompatible binders, can be consistent with those provided herein. In one aspect, biocompatible matrices may include calcium phosphate particles with or without biocompatible binders or a bone allograft, such as demineralized freeze-dried bone allograft (DFDBA), mineralized freeze-dried bone allograft (FDBA), or particulate demineralized bone matrix (DBM), or a bone xenograft or a combination thereof.

In another aspect, the present invention provides a composition useful to treat, prevent or delay the progression of ONJ or ORNJ, comprising a PDGF solution in a pharmaceutically acceptable buffer.

In another aspect, the present invention provides a kit comprising a biocompatible matrix in a first container and a solution comprising PDGF in a second container, wherein the kit is useful for treating or preventing ONJ or ORNJ. In another embodiment, the kit comprises a solution comprising PDGF in a pharmaceutically acceptable buffer in a container, wherein the kit is useful for treating or preventing ONJ or ORNJ. In another embodiment, the kit comprises a pharmaceutically acceptable buffer in a first container and a second container comprising PDGF, wherein the kit is useful for treating or preventing ONJ or ORNJ. In some embodiments, the solution comprises a predetermined concentration of PDGF. The concentration of the PDGF can be predetermined according to the surgical procedure being performed, such as treating, preventing or retarding the progression of ONJ or ORNJ. Moreover, in some embodiments, the biocompatible matrix can be present in the kit in a predetermined amount. The amount of biocompatible matrix provided by a kit can be dependent on the surgical procedure being performed. In some embodiments, the second container containing the PDGF solution comprises a syringe. A syringe can facilitate disposition of the PDGF solution in the biocompatible matrix. The kit optionally contains instructions for its use.

The present invention additionally provides methods for producing compositions useful to treat or prevent ONJ or ORNJ. In one embodiment, a method for producing such compositions comprises providing a solution comprising PDGF, providing a biocompatible matrix, and disposing the solution in the biocompatible matrix.

In another aspect, the present invention provides methods to treat ONJ, comprising providing a composition comprising a PDGF solution disposed in a biocompatible matrix and applying the composition to at least one site of an injury or defect in the maxilla or mandible and/or associated soft tissue.

In another aspect, the present invention provides methods to prevent ONJ or to retard the progression of ONJ, comprising providing a composition comprising a PDGF solution disposed in a biocompatible matrix and applying the composition to at least one site in the jaw vulnerable to ONJ.

In another aspect, the present invention provides methods to treat ORNJ, comprising providing a composition comprising a PDGF solution disposed in a biocompatible matrix and applying the composition to at least one site of an injury or defect in the maxilla or mandible and/or associated soft tissue.

In another aspect, the present invention provides methods to prevent ORNJ or to retard the progression of ORNJ, comprising providing a composition comprising a PDGF solution disposed in a biocompatible matrix and applying the composition to at least one site in the jaw vulnerable to ORNJ.

Accordingly, it is an object of the present invention to provide a composition comprising PDGF disposed in a biocompatible matrix useful to treat ONJ.

Another object of the present invention is to provide a composition comprising PDGF disposed in a biocompatible matrix useful to prevent the progression of or retard the progression of ONJ.

Yet another object of the present invention is to provide a composition comprising PDGF disposed in a biocompatible matrix useful to treat ORNJ.

Another object of the present invention is to provide a composition comprising PDGF disposed in a biocompatible matrix useful to prevent the progression of or retard the progression of ORNJ.

Yet another object of the present invention is to provide a method useful to treat ONJ comprising administration of a composition comprising PDGF disposed in a biocompatible matrix.

Another object of the present invention is to provide a method useful to prevent or slow the progression of ONJ comprising administration of a composition comprising PDGF disposed in a biocompatible matrix.

Yet another object of the present invention is to provide a method useful to treat ORNJ comprising administration of a composition comprising PDGF disposed in a biocompatible matrix.

Another object of the present invention is to provide a method useful to prevent or slow the progression of ORNJ comprising administration of a composition comprising PDGF disposed in a biocompatible matrix.

In another aspect, the present invention provides methods to treat ONJ, comprising providing a composition comprising a solution of PDGF in a pharmaceutically acceptable buffer and applying the composition to at least one site of an injury or defect in the maxilla or mandible and/or associated soft tissue.

In another aspect, the present invention provides methods to prevent ONJ or to retard the progression of ONJ, comprising providing a composition comprising a solution of PDGF in a pharmaceutically acceptable buffer and applying the composition to at least one site in the jaw vulnerable to ONJ.

In another aspect, the present invention provides methods to treat ORNJ, comprising providing a composition a solution of PDGF in a pharmaceutically acceptable buffer and applying the composition to at least one site of an injury or defect in the maxilla or mandible and/or associated soft tissue.

In another aspect, the present invention provides methods to prevent ORNJ or to retard the progression of ORNJ, comprising providing a composition comprising a solution of PDGF in a pharmaceutically acceptable buffer and applying the composition to at least one site in the jaw vulnerable to ORNJ.

Accordingly, it is an object of the present invention to provide a composition comprising a solution of PDGF in a pharmaceutically acceptable buffer useful to treat ONJ.

Another object of the present invention is to provide a composition comprising a solution of PDGF in a pharmaceutically acceptable buffer useful to prevent the progression of or retard the progression of ONJ.

Yet another object of the present invention is to provide a composition comprising a solution of PDGF in a pharmaceutically acceptable buffer useful to treat ORNJ.

Another object of the present invention is to provide a composition comprising a solution of PDGF in a pharmaceutically acceptable buffer useful to prevent the progression of or retard the progression of ORNJ.

Yet another object of the present invention is to provide a method useful to treat ONJ comprising administration of a composition comprising a solution of PDGF in a pharmaceutically acceptable buffer.

Another object of the present invention is to provide a method useful to prevent or slow the progression of ONJ comprising administration of a composition a solution of PDGF in a pharmaceutically acceptable buffer.

Yet another object of the present invention is to provide a method useful to treat ORNJ comprising administration of a composition comprising a solution of PDGF in a pharmaceutically acceptable buffer.

Another object of the present invention is to provide a method useful to prevent or slow the progression of ORNJ comprising administration of a composition a solution of PDGF in a pharmaceutically acceptable buffer.

These and other embodiments of the present invention are described in greater detail in the detailed description which follows. These and other objects, features and advantages of the present invention will become apparent after a review of the following detailed description of the disclosed embodiments and claims.

### DETAILED DESCRIPTION

The present invention relates to compositions and methods for treating, preventing and retarding the progression of ONJ and ORNJ. It is to be understood that the word jaw, when referring to ONJ or ORNJ, includes the mandible, the maxilla, other bones of the oral cavity and/or associated soft tissue. The present invention provides for the use of PDGF in a pharmaceutically acceptable vehicle in the preparation of a medicament for treating, preventing, or slowing the progression of ONJ or ORNJ in a patient. The present invention also provides for the use of composition comprising a solution of PDGF in a pharmaceutically acceptable vehicle disposed in a biocompatible matrix in the preparation of a medicament for treating, preventing, or slowing the progression of ONJ or ORNJ in a patient.

In one embodiment, for example, a composition for treating, preventing or retarding the progression of ONJ or ORNJ comprises a solution comprising PDGF and a biocompatible matrix, wherein the solution is disposed in the biocompatible matrix. In another embodiment, the composition comprises a solution of PDGF in a pharmaceutically acceptable buffer. In another embodiment, the composition comprises a PDGF solution disposed in a biocompatible matrix, wherein the biocompatible matrix comprises a scaffolding material and a biocompatible binder. In one aspect, biocompatible matrices may include calcium phosphate particles with or without biocompatible binders, or bone xenograft, or bone allograft, such as demineralized freeze-dried bone allograft (DFDBA), mineralized freeze-dried bone allograft (FDBA), or particulate demineralized bone matrix (DBM) or a combination thereof. In another embodiment, the biocompatible binder is collagen.

The present invention also provides kits useful for the prevention and treatment of ONJ and ORNJ. In another aspect, the present invention provides a kit comprising a first container comprising a biocompatible matrix and a solution comprising PDGF in a second container. The second container may act as a dispensing means, such as a syringe. In some embodiments, the solution comprises a predetermined concentration of PDGF. In another embodiment, the kit comprises a solution comprising PDGF in a pharmaceutically acceptable buffer in a container, wherein the kit is useful for treating or preventing ONJ or ORNJ. In another embodiment, the kit comprises a pharmaceutically acceptable buffer in a first container and a second container comprising PDGF, wherein the kit is useful for treating or preventing ONJ or ORNJ. In some embodiments, the concentration of PDGF is consistent with the values provided herein. The concentration of PDGF can be predetermined according to the condition being treated. The kit may further comprise a bone scaffolding material and the bone scaffolding material may further comprise a biocompatible binder. Moreover, the amount of biocompatible matrix provided by a kit can be dependent on the nature or classification of the bone defect being treated. Moreover, in some embodiments, the biocompatible matrix can be present in the kit in a predetermined amount. The amount of biocompatible matrix provided by a kit can be dependent on the surgical procedure being performed. Biocompatible matrix that may be included in the kit may be a bone scaffolding material, a bone scaffolding material and a biocompatible binder, and/or bone allograft such as DFDBA, FDBA or DBM, and/or bone xenograft, or a combination thereof. In some embodiments the biocompatible matrix comprises a β-tricalcium phosphate (β-TCP). In some embodiments the biocompatible matrix comprises a β-tricalcium phosphate with a binder such as collagen. A syringe can facilitate disposition of the PDGF solution in the biocompatible matrix for application at a desired site, such as a site in the jaw. The kit may also contain instructions for use.

Turning now to components that can be included in various embodiments of the present invention, compositions of the present invention comprise a solution comprising PDGF.

### PDGF Solutions

In one aspect, a composition provided by the present invention comprises a solution comprising PDGF and a biocompatible matrix, wherein the solution is disposed in the biocompatible matrix. In another aspect, a composition provided by the present invention comprises a solution of PDGF in a pharmaceutically acceptable buffer. In some embodiments, PDGF is present in the solution in a concentration ranging from about 0.01 mg/ml to about 10 mg/ml, from about 0.05 mg/ml to about 5 mg/ml, or from about 0.1 mg/ml to about 1.0 mg/ml. PDGF may be present in the solution at any concentration within these stated ranges. In other embodiments, PDGF is present in the solution at any one of the following concentrations: about 0.05 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; about 1.0 mg/ml; or about 3.0 mg/ml. In some embodiments, PDGF is present in the solution in a concentration raging from about 0.2 mg/ml to about 2 mg/ml, from about 0.3 mg/ml to about 3 mg/ml, from about 0.4 mg/ml to about 4 mg/ml, or from about 0.5 mg/ml to about 5 mg/ml. It is to be understood that these concentrations are simply examples of particular embodiments, and that the concentration of PDGF may be within any of the concentration ranges stated above.

Various amounts of PDGF may be used in the compositions of the present invention. Any clinically effective amount may be used. Amounts of PDGF that could be used include amounts in the following ranges: about 1 ug to about 50 mg, about 10 ug to about 25 mg, about 100 ug to about 10 mg, and about 250 ug to about 5 mg.

The concentration of PDGF or other growth factors in embodiments of the present invention can be determined by using an enzyme-linked immunoassay as described in U.S. Pat. Nos. 6,221,625, 5,747,273, and 5,290,708, or any other assay known in the art for determining PDGF concentration. When provided herein, the molar concentration of PDGF is determined based on the molecular weight of PDGF dimer (e.g., PDGF-BB; MW about 25 kDa).

In embodiments of the present invention, PDGF comprises PDGF homodimers and heterodimers, including PDGF-AA, PDGF-BB, PDGF-AB, PDGF-CC, PDGF-DD, and mixtures and derivatives thereof. In one embodiment, PDGF comprises PDGF-BB. In another embodiment PDGF comprises a recombinant human PDGF, such as rhPDGF-BB. In some embodiments, PDGF comprises mixtures of the various homodimers and/or heterodimers. Embodiments of the present invention contemplate any combination of PDGF-AA, PDGF-BB, PDGF-AB, PDGF-CC and/or PDGF-DD.

PDGF, in some embodiments, can be obtained from natural sources. In other embodiments, PDGF can be produced by recombinant DNA techniques. In other embodiments, PDGF or fragments thereof may be produced using peptide synthesis techniques known to one of ordinary skill in the art, such as solid phase peptide synthesis. When obtained from natural sources, PDGF can be derived from biological fluids. Biological fluids, according to some embodiments, can comprise any treated or untreated fluid associated with living organisms including blood

Biological fluids, in another embodiment, can also comprise blood components including platelet concentrate (PC), apheresed platelets, platelet-rich plasma (PRP), plasma, serum, fresh frozen plasma (FFP), and buffy coat (BC). Biological fluids, in a further embodiment, can comprise platelets separated from plasma and resuspended in a physiological fluid.

When produced by recombinant DNA techniques, a DNA sequence encoding a single monomer (e.g., PDGF B-chain or A-chain), or a fragment thereof, in some embodiments, can be inserted into cultured prokaryotic cells or eukaryotic cells for expression to subsequently produce the homodimer (e.g. PDGF-BB or PDGF-AA). In other embodiments, a PDGF heterodimer can be generated by inserting DNA sequences encoding for both monomeric units of the heterodimer into cultured prokaryotic, eukaryotic, or insect cells and allowing the translated monomeric units to be processed by the cells to produce the heterodimer (e.g. PDGF-AB). Commercially available cGMP recombinant PDGF-BB can be obtained commercially from Novartis Corporation (Chiron) (Emeryville, Calif.). Research grade rhPDGF-BB can be obtained from multiple sources including R&D Systems, Inc. (Minneapolis, Minn.), BD Biosciences (San Jose, Calif.), and Chemicon International (Temecula, Calif.). In some embodiments, monomeric units can be produced in prokaryotic cells in a denatured form, wherein the denatured form is subsequently refolded into an active molecule.

In embodiments of the present invention, PDGF comprises one or more PDGF fragments. In one embodiment recombinant human (rh) PDGF-B comprises the following fragments: amino acid sequences 1-31, 1-32, 33-108, 33-109, and/or 1-108 of the entire B chain, or a mixture thereof. The complete amino acid sequence (1-109) of the B chain of PDGF is provided in FIG. 15 of U.S. Pat. No. 5,516,896. It is to be understood that the rhPDGF compositions, in some embodiments of the present invention, can comprise a combination of intact rhPDGF-B (1-109) and fragments thereof. Other fragments of PDGF may be employed such as those disclosed in U.S. Pat. No. 5,516,896. In accordance with one embodiment, the rhPDGF-BB comprises at least 60% of intact rhPDGF-B (1-109). In another embodiment, the rhPDGF-BB comprises at least 65%, 75%, 80%, 85%, 90%, 95% or 99% of intact rhPDGF-B (1-109).

In some embodiments of the present invention, PDGF can be purified. Purified PDGF, as used herein, comprises compositions having greater than about 95% by weight PDGF prior to incorporation in solutions of the present invention. The solution may be any pharmaceutically acceptable solution. In other embodiments, the PDGF can be substantially purified. Substantially purified PDGF, as used herein, comprises compositions having about 5% to about 95% by weight PDGF prior to incorporation into solutions of the present invention. In one embodiment, substantially purified PDGF comprises compositions having about 65% to about 95% by weight PDGF prior to incorporation into solutions of the present invention. In other embodiments, substantially purified PDGF comprises compositions having about 70% to about 95%, about 75% to about 95%, about 80% to about 95%, about 85% to about 95%, or about 90% to about 95%, by weight PDGF, prior to incorporation into solutions of the present invention. Purified PDGF and substantially purified PDGF may be incorporated into scaffolds and binders.

In a further embodiment, PDGF can be partially purified. Partially purified PDGF, as used herein, comprises compositions having PDGF in the context of platelet rich plasma (PRP), fresh frozen plasma (FFP), or any other blood product that requires collection and separation to produce PDGF. Embodiments of the present invention contemplate that any of the PDGF isoforms provided herein, including homodimers and heterodimers, can be purified or partially purified. Compositions of the present invention containing PDGF mixtures may contain PDGF isoforms or PDGF fragments in partially purified proportions. Partially purified and purified PDGF, in some embodiments, can be prepared as described in U.S. patent application Ser. No. 11/159,533 (Publication No: 20060084602).

In some embodiments, solutions comprising PDGF are formed by solubilizing PDGF in one or more buffers. Buffers suitable for use in PDGF solutions of the present invention can comprise, but are not limited to, carbonates, phosphates (e.g. phosphate buffered saline), histidine, acetates (e.g. sodium acetate), acidic buffers such as acetic acid and HCl, and organic buffers such as lysine, Tris buffers (e.g. tris(hydroxymethyl)aminoethane), N-2-hydroxyethylpiperazine-N′-2-ethanesulfonic acid (HEPES), and 3-(N-morpholino) propanesulfonic acid (MOPS). Buffers can be selected based on biocompatibility with PDGF and the buffer's ability to impede undesirable protein modification. Buffers can additionally be selected based on compatibility with host tissues. In another aspect, when the composition provided by the present invention comprises a solution of PDGF in a pharmaceutically acceptable buffer, any pharmaceutically acceptable buffer may be employed as known to one of ordinary skill in the art. In one embodiment, sodium acetate buffer is used. The buffers may be employed at different molarities, for example about 0.1 mM to about 100 mM, about 1 mM to about 50 mM, about 5 mM to about 40 mM, about 10 mM to about 30 mM, or about 15 mM to about 25 mM, or any molarity within these ranges. In one embodiment, an acetate buffer is employed at a molarity of about 20 mM.

In another embodiment, solutions comprising PDGF are formed by solubilizing lyophilized PDGF in water, wherein prior to solubilization the PDGF is lyophilized from an appropriate buffer.

Solutions comprising PDGF, according to embodiments of the present invention, can have a pH ranging from about 3.0 to about 8.0. In one embodiment, a solution comprising PDGF has a pH ranging from about 5.0 to about 8.0, more preferably about 5.5 to about 7.0, most preferably about 5.5 to about 6.5, or any value within these ranges. The pH of solutions comprising PDGF, in some embodiments, can be compatible with the prolonged stability and efficacy of PDGF or any other desired biologically active agent. PDGF is generally more stable in an acidic environment. Therefore, in accordance with one embodiment the present invention comprises an acidic storage formulation of a PDGF solution. In accordance with this embodiment, the PDGF solution preferably has a pH from about 3.0 to about 7.0, and more preferably from about 4.0 to about 6.5. The biological activity of PDGF, however, can be optimized in a solution having a neutral pH range. Therefore, in a further embodiment, the present invention comprises a neutral pH formulation of a PDGF solution. In accordance with this embodiment, the PDGF solution preferably has a pH from about 5.0 to about 8.0, more preferably about 5.5 to about 7.0, most preferably about 6.0 to about 7.0. In accordance with a method of the present invention, an acidic PDGF solution is reformulated to a neutral pH composition, wherein such composition is then used to treat or prevent ONJ or ORNJ. In accordance with a preferred embodiment of the present invention, the PDGF utilized in the solutions is rhPDGF-BB.

In some embodiments, the pH of the PDGF containing solution may altered to optimize the binding kinetics of PDGF to a matrix substrate or linker. If desired, as the pH of the material equilibrates to adjacent material, the bound PDGF may become labile.

The pH of solutions comprising PDGF, in some embodiments, can be controlled by the buffers recited herein. Various proteins demonstrate different pH ranges in which they are stable. Protein stabilities are primarily reflected by isoelectric points and charges on the proteins. The pH range can affect the conformational structure of a protein and the susceptibility of a protein to proteolytic degradation, hydrolysis, oxidation, and other processes that can result in modification to the structure and/or biological activity of the protein. Any pharmaceutically acceptable buffer known to one of ordinary skill in the art may be used to form solutions of PDGF for administration to the recipient.

In some embodiments, solutions comprising PDGF can further comprise additional components, such as other biologically active agents. In other embodiments, solutions comprising PDGF can further comprise cell culture media, other stabilizing proteins such as albumin, antibacterial agents, protease inhibitors [e.g., ethylenediaminetetraacetic acid (EDTA), ethylene glycol-bis(beta-aminoethylether)-N,N,N′,N′-tetraacetic acid (EGTA), aprotinin, ε-aminocaproic acid (EACA), etc.] and/or other growth factors such as fibroblast growth factors (FGFs), epidermal growth factors (EGFs), transforming growth factors (TGFs), keratinocyte growth factors (KGFs), insulin-like growth factors (IGFs), hepatocyte growth factors (HGFs), bone morphogenetic proteins (BMPs), or other PDGFs including compositions of PDGF-AA, PDGF-BB, PDGF-AB, PDGF-CC and/or PDGF-DD.

In addition to solutions comprising PDGF, compositions of the present invention also comprise a biocompatible matrix in which to dispose the PDGF solutions and may also comprise a biocompatible binder.

### Biocompatible Matrix

Scaffolding Material

A biocompatible matrix, according to embodiments of the present invention, comprises a scaffolding material. The scaffolding material, according to embodiments of the present invention, provides the framework or scaffold for new tissue and/or bone growth to occur. A scaffolding material, in some embodiments, comprises multi-directional and interconnected pores of varying diameters. In some embodiments, a scaffolding material comprises a plurality of pockets and non-interconnected pores of various diameters in addition to the interconnected pores. In some embodiments the biocompatible matrix comprises a xenograft, or an allograft such as DFDBA, FDBA or DBM, or a combination thereof. A scaffolding material, in some embodiments, comprises at least one calcium phosphate. In some embodiments biocompatible matrix comprises at least one calcium phosphate with a binder, for example collagen, and/or allograft. In other embodiments, a scaffolding material can comprise a plurality of calcium phosphates. Calcium phosphates suitable for use as a scaffolding material, in embodiments of the present invention, have a calcium to phosphorus atomic ratio ranging from 0.5 to 2.0.

Non-limiting examples of calcium phosphates suitable for use as scaffolding materials comprise amorphous calcium phosphate, monocalcium phosphate monohydrate (MCPM), monocalcium phosphate anhydrous (MCPA), dicalcium phosphate dihydrate (DCPD), dicalcium phosphate anhydrous (DCPA), octacalcium phosphate (OCP), α-tricalcium phosphate, β-tricalcium phosphate, hydroxyapatite (OHAp), poorly crystalline hydroxyapatite, tetracalcium phosphate (TTCP), heptacalcium decaphosphate, calcium metaphosphate, calcium pyrophosphate dihydrate, carbonated calcium phosphate, and calcium pyrophosphate, hydroxyapatite, or derivatives thereof.

In some embodiments, a scaffolding material comprises a polymeric material. A polymeric scaffold, in some embodiments, comprises collagen, polylactic acid, poly(L-lactide), poly(D,L-lactide), polyglycolic acid, poly(L-lactide-co-glycolide), poly(L-lactide-co-D,L-lactide), polyacrylate, polymethacrylate, polymethylmethacrylate, chitosan, or combinations or derivatives thereof.

In some embodiments, a scaffolding material comprises porous structure. Porous scaffolding materials, according to some embodiments, can comprise pores having diameters ranging from about 1 μm to about 1 mm. In one embodiment, a scaffolding material comprises macropores having diameters ranging from about 100 μm to about 1 mm or greater. In another embodiment, a scaffolding material comprises mesopores having diameters ranging from about 10 μm to about 100 μm. In a further embodiment, a scaffolding material comprises micropores having diameters less than about 10 μm. Embodiments of the present invention contemplate scaffolding materials comprising macropores, mesopores, and micropores or any combination thereof.

A porous scaffolding material, in one embodiment, has a porosity greater than about 25% or greater than about 40%. In another embodiment, a porous scaffolding material has a porosity greater than about 50%, greater than about 60%, greater than about 65%, greater than about 70%, greater than about 80%, or greater than about 85%. In a further embodiment, a porous scaffolding material has a porosity greater than about 90%. In some embodiments, a porous scaffolding material comprises a porosity that facilitates cell migration into the scaffolding material.

In some embodiments, a scaffolding material comprises a plurality of particles. Scaffolding particles may be mm, μm, or submicron (nm) in size. Scaffolding particles, in one embodiment, have an average diameter ranging from about 1 μm to about 5 mm. In other embodiments, particles have an average diameter ranging from about 1 mm to about 2 mm, from about 1 mm to about 3 mm, or from about 250 μm to about 750 μm. Scaffolding particles, in another embodiment, have an average diameter ranging from about 100 μm to about 300 μm. In a further embodiment, scaffolding particles have an average diameter ranging from about 75 μm to about 300 μm. In additional embodiments, scaffolding particles have an average diameter less than about 25 μm, less than about 1 μm, or less than about 1 mm.

In some embodiments, scaffolding particles have an average diameter ranging from about 100 μm to about 5 mm or from about 100 μm to about 3 mm. In other embodiments, scaffolding particles have an average diameter ranging from about 250 μm to about 2 mm, from about 250 μm to about 1 mm, or from about 200 μm to about 3 mm. Particles may also be in the range of about 1 nm to about 1 μm, less than about 500 nm, or less than about 250 nm.

Scaffolding materials, according to some embodiments, can be provided in a shape suitable for implantation (e.g., a sphere, a cylinder, or a block). In other embodiments, scaffolding materials are moldable, extrudable and/or injectable. Moldable, extrudable, and injectable scaffolding materials can facilitate efficient placement of compositions of the present invention in and around injuries and/or defects in the jaw. In some embodiments, moldable, extrudable, and/or injectable scaffolding materials are applied to sites in the jaw with a spatula or equivalent device. In some embodiments, scaffolding materials are flowable. Flowable scaffolding materials, in some embodiments, can be applied to sites in the jaw through a syringe and needle or cannula.

In some embodiments, scaffolding materials are bioresorbable. A scaffolding material, in one embodiment, can be at least 30%, 40%, 50%, 60%, 70%, 75%, or 90% resorbed within one year subsequent to in vivo implantation. In another embodiment, a scaffolding material can be at least 5%, 10%, 20%, 30%, 40%, 50%, 60%, 70%, 75%, or 90% resorbed within 1, 3, 6, 9, 12, or 18 months of in vivo implantation. In some embodiments, scaffolding materials are greater than 90% resorbed within 1, 3, 6, 9, 12, or 18 months of in vivo implantation. Bioresorbability will be dependent on: (1) the nature of the matrix material (i.e., its chemical make up, physical structure and size); (2) the location within the body in which the matrix is placed; (3) the amount of matrix material that is used; (4) the metabolic state of the patient (diabetic/non-diabetic, osteoporotic, smoker, old age, steroid use, etc.); (5) the extent and/or type of injury treated; and (6) the use of other materials in addition to the matrix such as other bone anabolic, catabolic and anti-catabolic factors.

### Scaffolding Comprising Allograft

A composition of the present invention, according to some embodiments, can further comprise grafting materials with PDGF, including autologous bone marrow, autologous platelet extracts, allografts, synthetic bone matrix materials, xenografts, and derivatives thereof and combinations thereof. In some embodiments the biocompatible matrix comprises an allograft such as DFDBA, FDBA or DBM or a combination thereof. Such allografts are available from commercial vendors and bone banks. Characteristics of the allograft include but are not limited to allogeneic bone particulates (various ranges, 125-500 μm; 125-1,000 μm), with particulates being either completely or substantially demineralized (˜4% residual is usually the most demineralization used) or non-demineralized, but deorganified, and combinations thereof. Likewise, deorganified xenogeneic materials may be employed, e.g., BioOss (Geistlich Biomaterials, Inc.). Allograft and xenograft characteristics can include particles, blends, meshes, blocks and varying amounts of demineralization and deorganification and combinations thereof.

### Scaffolding Comprising β-Tricalcium Phosphate

A scaffolding material for use as a biocompatible matrix, in some embodiments, comprises β-tricalcium phosphate (β-TCP). β-TCP, according to some embodiments, can comprise a porous structure having multidirectional and interconnected pores of varying diameters. In some embodiments, β-TCP comprises a plurality of pockets and non-interconnected pores of various diameters in addition to the interconnected pores. The porous structure of β-TCP, in one embodiment, comprises macropores having diameters ranging from about 100 μm to about 1 mm or greater, mesopores having diameters ranging from about 10 μm to about 100 μm, and micropores having diameters less than about 10 μm. Macropores and mesopores of the β-TCP can facilitate tissue in-growth including chondrocyte migration and proliferation as well as osteoinduction and osteoconduction while macropores, mesopores and micropores can permit fluid communication and nutrient transport to support tissue regrowth, including cartilage and/or bone regrowth, throughout the β-TCP biocompatible matrix.

In comprising a porous structure, β-TCP, in some embodiments, can have a porosity greater than 25% or greater than about 40%. In other embodiments, β-TCP can have a porosity greater than 50%, greater than about 60%, greater than about 65%, greater than about 70%, greater than about 75%, greater than about 80%, or greater than about 85%. In a further embodiment, β-TCP can have a porosity greater than 90%. In some embodiments, β-TCP can have a porosity that facilitates cell migration into the β-TCP.

In some embodiments, a scaffolding material comprises β-TCP particles, β-TCP particles, in some embodiments, can individually demonstrate any of the pore diameters, pore structures, and porosities provided herein for scaffolding materials.

β-TCP particles, in one embodiment have an average diameter ranging from about 1 μm to about 5 mm. In other embodiments, β-TCP particles have an average diameter ranging from about 1 mm to about 2 mm, from about 1 mm to about 3 mm, from about 100 μm to about 5 mm, from about 100 μm to about 3 mm, from about 250 μm to about 2 mm, from about 250 μm to about 750 μm, from about 250 μm to about 1 mm, from about 250 μm to about 2 mm, or from about 200 μm to about 3 mm. In another embodiment, β-TCP particles have an average diameter ranging from about 100 μm to about 300 μm. In some embodiments, β-TCP particles have an average diameter ranging from about 75 μm to about 300 μm. In some embodiments, β-TCP particles have an average diameter of less than about 25 μm, less than about 1 μm, or less than about 1 mm. In some embodiments, β-TCP particles have an average diameter ranging from about 1 nm to about 1 μm. In a further embodiment, β-TCP particles have an average diameter less than about 500 nm or less than about 250 nm.

A biocompatible matrix comprising a β-TCP scaffolding material, in some embodiments, is provided in a shape suitable for implantation (e.g., a sphere, a cylinder, or a block). In other embodiments, a β-TCP scaffolding material is moldable, extrudable, and/or flowable thereby facilitating application of the matrix to desired sites in the jaw. Flowable matrices may be applied through syringes, tubes, cannulas or spatulas.

A β-TCP scaffolding material, according to some embodiments, is bioresorbable. In one embodiment, a β-TCP scaffolding material can be at least 30%, 40%, 50%, 60%, 65%, 70%, 75%, 80%, or 85% resorbed one year subsequent to in vivo implantation. In another embodiment, a β-TCP scaffolding material can be greater than 90% resorbed one year subsequent to in vivo implantation.

### Scaffolding Material and Biocompatible Binder

In another embodiment, a biocompatible matrix comprises a scaffolding material and a biocompatible binder.

Biocompatible binders, according to some embodiments, can comprise materials operable to promote cohesion between combined substances. A biocompatible binder, for example, can promote adhesion between particles of a scaffolding material in the formation of a biocompatible matrix. In certain embodiments, the same material may serve as both a scaffolding material and a binder. In one embodiment, for example, polymeric materials described herein, such as collagen or chitosan, can serve as both a scaffolding material and a binder.

Biocompatible binders, in some embodiments, can comprise collagen, elastin, polysaccharides, nucleic acids, carbohydrates, proteins, polypeptides, poly(α-hydroxy acids), poly(lactones), poly(amino acids), poly(anhydrides), polyurethanes, poly(orthoesters), poly(anhydride-co-imides), poly(orthocarbonates), poly(α-hydroxy alkanoates), poly(dioxanones), poly(phosphoesters), polylactic acid, poly(L-lactide) (PLLA), poly(D,L-lactide) (PDLLA), polyglycolide (PGA), poly(lactide-co-glycolide (PLGA), poly(L-lactide-co-D,L-lactide), poly(D,L-lactide-co-trimethylene carbonate), polyglycolic acid, polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide, and copolymers and mixtures thereof.

Biocompatible binders, in other embodiments, can comprise alginic acid, arabic gum, guar gum, xantham gum, gelatin, chitin, chitosan, chitosan acetate, chitosan lactate, chondroitin sulfate, N,O-carboxymethyl chitosan, a dextran (e.g., α-cyclodextrin, β-cyclodextrin, γ-cyclodextrin, or sodium dextran sulfate), fibrin glue, lecithin, phosphatidylcholine derivatives, glycerol, hyaluronic acid, sodium hyaluronate, a cellulose (e.g., methylcellulose, carboxymethylcellulose, hydroxypropyl methylcellulose, or hydroxyethyl cellulose), a glucosamine, a proteoglycan, a starch (e.g., hydroxyethyl starch or starch soluble), lactic acid, pluronic acids, sodium glycerophosphate, glycogen, a keratin, silk, and derivatives and mixtures thereof.

In some embodiments, a biocompatible binder is water-soluble. A water-soluble binder can dissolve from the biocompatible matrix shortly after its implantation, thereby introducing macroporosity into the biocompatible matrix. Macroporosity, as discussed herein, can increase the osteoconductivity of the implant material by enhancing the access and, consequently, the remodeling activity of the osteoclasts and osteoblasts at the implant site.

In some embodiments, a biocompatible binder can be present in a biocompatible matrix in an amount ranging from about 5 weight percent to about 50 weight percent of the matrix. In other embodiments, a biocompatible binder can be present in an amount ranging from about 10 weight percent to about 40 weight percent of the biocompatible matrix. In another embodiment, a biocompatible binder can be present in an amount ranging from about 15 weight percent to about 35 weight percent of the biocompatible matrix. In a further embodiment, a biocompatible binder can be present in an amount of about 20 weight percent of the biocompatible matrix. In another embodiment, a biocompatible binder can be present in a biocompatible matrix in an amount greater than about 50 weight percent or greater than about 60 weight percent of the matrix. In one embodiment, a biocompatible binder can be present in a biocompatible matrix in an amount up to about 99 weight percent of the matrix.

A biocompatible matrix comprising a scaffolding material and a biocompatible binder, according to some embodiments, can be flowable, moldable, and/or extrudable. In such embodiments, a biocompatible matrix can be in the form of a paste, putty or a granular form. A biocompatible matrix in the form of a paste or putty, in one embodiment, can comprise particles of a scaffolding material adhered to one another by a biocompatible binder.

A biocompatible matrix in paste or putty form can be molded into the desired implant shape or can be molded to the contours of the implantation site. In one embodiment, a biocompatible matrix in paste or putty form can be injected into an implantation site with a syringe or cannula.

In some embodiments, a biocompatible matrix in paste or putty form does not harden and retains a flowable and moldable form subsequent to implantation. In other embodiments, a paste or putty can harden subsequent to implantation, thereby reducing matrix flowability and moldability.

A biocompatible matrix comprising a scaffolding material and a biocompatible binder, in some embodiments, can also be provided in a predetermined shape including a block, sphere, or cylinder or any desired shape, for example a shape defined by a mold or a site of application.

A biocompatible matrix comprising a scaffolding material and a biocompatible binder, in some embodiments, is bioresorbable. A biocompatible matrix, in such embodiments, can be resorbed within one year of in vivo implantation. In another embodiment, a biocompatible matrix comprising a scaffolding material and a biocompatible binder can be resorbed within 1, 3, 6, or 9 months of in vivo implantation. In some embodiments, a biocompatible matrix comprising a scaffolding material and a biocompatible binder can be resorbed within 1, 3, or 6 years of in vivo implantation. Bioresorbability will be dependent on: (1) the nature of the matrix material (i.e., its chemical make up, physical structure and size); (2) the location within the body in which the matrix is placed; (3) the amount of matrix material that is used; (4) the metabolic state of the patient (diabetic/non-diabetic, osteoporotic, smoker, old age, steroid use, etc.); (5) the extent and/or type of injury treated; and (6) the use of other materials in addition to the matrix such as other bone anabolic, catabolic and anti-catabolic factors.

### Biocompatible Matrix Comprising β-TCP and Collagen

In some embodiments, a biocompatible matrix can comprise a β-TCP scaffolding material and a biocompatible collagen binder. β-TCP scaffolding materials suitable for combination with a collagen binder are consistent with those provided hereinabove.

A collagen binder, in some embodiments, comprises any type of collagen, including Type I, Type II, and Type III collagens. In one embodiment, a collagen binder comprises a mixture of collagens, such as a mixture of Type I and Type II collagen. In other embodiments, a collagen binder is soluble under physiological conditions. Other types of collagen present in bone or musculoskeletal tissues may be employed. Recombinant, synthetic and naturally occurring forms of collagen may be used in the present invention.

A biocompatible matrix, according to some embodiments, can comprise a plurality of β-TCP particles adhered to one another with a collagen binder. In some embodiments, β-TCP particles for combination with a collagen binder have an average diameter ranging from about 1 μm to about 5 mm. In other embodiments, β-TCP particles have an average diameter ranging from about 1 mm to about 2 mm, from about 1 mm to about 3 mm, from about 100 μm to about 5 mm, from about 100 μm to about 3 mm, from about 250 μm to about 2 mm, from about 250 μm to about 750 μm, from about 250 μm to about 1 mm, from about 250 μm to about 2 mm, or from about 200 μm to about 3 mm. In another embodiment, β-TCP particles have an average diameter ranging from about 100 μm to about 300 μm. In some embodiments, β-TCP particles have an average diameter ranging from about 75 μm to about 300 μm. In some embodiments, β-TCP particles have an average diameter of less than about 25 μm, less than about 1 μm, or less than about 1 mm. In some embodiments, β-TCP particles have an average diameter ranging from about 1 nm to about 1 μm. In a further embodiment, β-TCP particles have an average diameter less than about 500 nm or less than about 250 nm.

β-TCP particles, in some embodiments, can be adhered to one another by the collagen binder so as to produce a biocompatible matrix having a porous structure. In some embodiments, the porous structure of the biocompatible matrix comprising β-TCP particles and a collagen binder demonstrates multidirectional and interconnected pores of varying diameters. In some embodiments, the biocompatible matrix comprises a plurality of pockets and non-interconnected pores of various diameters in addition to the interconnected pores.

In some embodiments, a biocompatible matrix comprising β-TCP particles and a collagen binder can comprise pores having diameters ranging from about 1 μm to about 1 mm or greater. A biocompatible matrix comprising β-TCP particles and a collagen binder can comprise macropores having diameters ranging from about 100 μm to about 1 mm, mesopores having diameters ranging from about 10 μm to 100 μm, and micropores having diameters less than about 10 μm.

A biocompatible matrix comprising β-TCP particles and a collagen binder can have a porosity greater than about 25% or greater than about 40%. In another embodiment, the biocompatible matrix can have a porosity greater than about 50%, greater than about 65%, greater than about 70%, greater than about 75%, greater than about 80%, or greater than about 85%. In a further embodiment, the biocompatible matrix can have a porosity greater than about 90%. In some embodiments, the biocompatible matrix can have a porosity that facilitates cell migration into the matrix.

In some embodiments, the β-TCP particles, can individually demonstrate any of the pore diameters, pore structures, and porosities provided herein for a biocompatible matrix comprising β-TCP and collagen binder.

A biocompatible matrix comprising β-TCP particles, in some embodiments, can comprise a collagen binder in an amount ranging from about 5 weight percent to about 50 weight percent of the matrix. In other embodiments, a collagen binder can be present in an amount ranging from about 10 weight percent to about 40 weight percent of the biocompatible matrix. In another embodiment, a collagen binder can be present in an amount ranging from about 15 weight percent to about 35 weight percent of the biocompatible matrix. In a further embodiment, a collagen binder can be present in an amount of about 20 weight percent of the biocompatible matrix.

A biocompatible matrix comprising β-TCP particles and a collagen binder, according to some embodiments, can be flowable, moldable, and/or extrudable. In such embodiments, the biocompatible matrix can be in the form of a paste or putty. A paste or putty can be molded into the desired implant shape or can be molded to the contours of the implantation site. In one embodiment, a biocompatible matrix in paste or putty or granular form comprising β-TCP particles and a collagen binder can be injected into an implantation site with a syringe or cannula.

In some embodiments, a biocompatible matrix in paste or putty form comprising β-TCP particles and a collagen binder can retain a flowable and moldable form when implanted. In other embodiments, the paste or putty can harden subsequent to implantation, thereby reducing matrix flowability and moldability.

A biocompatible matrix comprising β-TCP particles and a collagen binder, in some embodiments, can be provided in a predetermined shape such as a block, sphere, or cylinder.

A biocompatible matrix comprising β-TCP particles and a collagen binder can be resorbable. In one embodiment, a biocompatible matrix comprising β-TCP particles and a collagen binder can be at least 75% resorbed one year subsequent to in vivo implantation. In another embodiment, a biocompatible matrix comprising β-TCP particles and a collagen binder can be greater than 90% resorbed one year subsequent to in vivo implantation.

In some embodiments, a solution comprising PDGF can be disposed in a biocompatible matrix to produce a composition for treating, preventing or slowing the progression of ONJ or ORNJ. In some embodiments, a solution comprising PDGF can be disposed in a biocompatible matrix for treating bone.

### Disposing PDGF Solution in a Biocompatible Matrix

The present invention provides methods for producing compositions for treating or preventing ONJ or ORNJ. In one embodiment, a method for producing such compositions comprises providing a solution comprising PDGF, providing a biocompatible matrix, and disposing the solution in the biocompatible matrix. PDGF solutions and biocompatible matrices suitable for combination are consistent with those described hereinabove.

In some embodiments, a PDGF solution can be disposed in a biocompatible matrix by soaking the biocompatible matrix in the PDGF solution. A PDGF solution, in another embodiment, can be disposed in a biocompatible matrix by injecting the biocompatible matrix with the PDGF solution. In some embodiments, injecting a PDGF solution can comprise disposing the PDGF solution in a syringe and expelling the PDGF solution into the biocompatible matrix to saturate the biocompatible matrix.

In some embodiments, the PDGF is absorbed into the pores of the biocompatible matrix. In some embodiments, the PDGF is adsorbed onto one or more surfaces of the biocompatible matrix, including surfaces within pores of the biocompatible matrix.

The biocompatible matrix, according to some embodiments, can be in a predetermined shape, such as a brick or cylinder, prior to receiving a PDGF solution. Subsequent to receiving a PDGF solution, the biocompatible matrix can have a paste or putty form that is flowable, extrudable, and/or injectable. In other embodiments, the biocompatible matrix can already demonstrate a flowable, extrudable, and/or injectable paste or putty form prior to receiving a solution comprising PDGF.

### Compositions Further Comprising Biologically Active Agents

Compositions of the present invention, according to some embodiments, can further comprise one or more biologically active agents in addition to PDGF. Biologically active agents that can be incorporated into compositions of the present invention, in addition to PDGF, can comprise organic molecules, inorganic materials, proteins, peptides, nucleic acids (e.g., genes, gene fragments, small-interfering ribonucleic acids [si-RNAs] gene regulatory sequences, nuclear transcriptional factors, and antisense molecules), nucleoproteins, polysaccharides (e.g., heparin), glycoproteins, and lipoproteins. Non-limiting examples of biologically active compounds that can be incorporated into compositions of the present invention, including, e.g., anti-cancer agents, antibiotics, analgesics, anti-inflammatory agents, immunosuppressants, enzyme inhibitors, antihistamines, hormones, muscle relaxants, prostaglandins, trophic factors, osteoinductive proteins, growth factors, and vaccines, are disclosed in U.S. patent application Ser. No. 11/159,533 (Publication No: 20060084602). Biologically active compounds that can be incorporated into compositions of the present invention include osteoinductive factors such as insulin-like growth factors, fibroblast growth factors, or other PDGFs. In accordance with other embodiments, biologically active compounds that can be incorporated into compositions of the present invention preferably include osteoinductive and osteostimulatory factors such as bone morphogenetic proteins (BMPs), BMP mimetics, calcitonin, or calcitonin mimetics, statins, statin derivatives, fibroblast growth factors, insulin-like growth factors, growth-differentiating factors, small molecule or antibody blockers of Wnt antagonists (e.g. sclerostin, DKK, soluble Wnt receptors) or parathyroid hormone. In some embodiments, factors also include protease inhibitors, as well as osteoporotic treatments that decrease bone resorption including bisphosphonates, teriparadide, and antibodies to the activator receptor of the NF-kB ligand (RANK) ligand.

Standard protocols and regimens for delivery of additional biologically active agents are known in the art. Additional biologically active agents can be introduced into compositions of the present invention in amounts that allow delivery of an appropriate dosage of the agent to the implant site. In most cases, dosages are determined using guidelines known to practitioners and applicable to the particular agent in question. The amount of an additional biologically active agent to be included in a composition of the present invention can depend on such variables as the type and extent of the condition, the overall health status of the particular patient, the formulation of the biologically active agent, release kinetics, and the bioresorbability of the biocompatible matrix. Standard clinical trials may be used to optimize the dose and dosing frequency for any particular additional biologically active agent.

A composition of the present invention, according to some embodiments, can further comprise the addition of additional grafting materials with PDGF including autologous bone marrow, autologous platelet extracts, allografts, synthetic bone matrix materials, xenografts, and derivatives thereof.

### Administering the Compositions of the Present Invention for Treating, Preventing or Slowing the Progression of ONJ or ORNJ

The compositions may be administered through any appropriate means. In one embodiment, administration of the composition comprising the biocompatible matrix containing PDGF may occur through direct application of the composition at the desired site. In another embodiment, administration of the composition comprising a solution of PDGF in a pharmaceutically acceptable carrier may occur through direct application of the composition at the desired site. Such sites include, but are not limited to, the maxilla, the mandible and their adnexia which includes the alveolar structures, and any other bone or soft tissues affected by ONJ or ORNJ. In the mandible, sites anterior to the retromolar pad may constitute a desired site. For example, when a surgical field is open in the maxilla or mandible of a patient with ONJ or ORNJ, and a necrotic site is debrided and prepared, the composition may be applied through a syringe delivery, through a needle or cannula, by direct application with a spatula, forceps, spoon or other acceptable means. In other embodiments, when a site predicted to be vulnerable to ONJ or ORNJ is identified, the site may be exposed surgically and the composition applied, or the composition may be applied by syringe and needle injection through the skin to the vicinity of the desired site without surgically exposing the site in the mandible or maxilla. In other embodiments, the composition may be applied to the desired site through direct percutaneous administration.

In some embodiments, the PDGF-containing composition is administered concurrently with the dental procedure or shortly after the dental procedure. For example, a patient at risk and having a dental surgical procedure such as an extraction has the PDGF-containing composition, in one embodiment, co-administered with, for example, a dental extraction medicament or dressing. Yet another example for the PDGF-containing composition is an oro-dental cystrectomy where the PDGF-containing composition is placed into the cystic cavity. Yet another example includes a periodontal procedure where gingival tissues were incised and alveolar and/or inter-radicular osseo-dental surgery were performed and the PDGF-containing composition is co-administered with the periodontal therapy dressing.

In some embodiments, the quantity of the PDGF-containing composition administered is determined by the bone volume that had been surgically removed, for example from an extraction socket, a cystrectomy, or during periodontal bone surgery.

In some embodiments, for the prophylactic treatment of ORNJ or ONJ with a PDGF containing composition, radiographic determination of a thickening of the periodontal ligament in addition to the clinical signs and symptoms noted earlier, may be considered a diagnostic criterion.

### Kits

In another aspect, the present invention provides a kit comprising a first container comprising a biocompatible matrix and solution comprising PDGF in a second container, wherein the kit is useful for treating or preventing ONJ or ORNJ. In some embodiments, the solution comprises a predetermined concentration of PDGF. In another embodiment, the kit comprises a solution comprising PDGF in a pharmaceutically acceptable buffer in a container, wherein the kit is useful for treating or preventing ONJ or ORNJ. In another embodiment, the kit comprises a pharmaceutically acceptable buffer in a first container and a second container comprising PDGF, wherein the kit is useful for treating or preventing ONJ or ORNJ. The concentration of PDGF can be predetermined according to the nature or classification of the fracture being treated. The kit may further comprise a bone scaffolding material and the bone scaffolding material may further comprise a biocompatible binder. Moreover, the amount of biocompatible matrix provided by a kit can be dependent on the nature or classification of the bone being treated. Biocompatible matrix that may be included in the kit may be a bone scaffolding material, a bone scaffolding material and a biocompatible binder, and/or bone allograft such as DFDBA or DBM. In one embodiment the bone scaffolding material comprises a calcium phosphate, such as β-TCP. In some embodiments, the second container containing the PDGF solution comprises a syringe. A syringe can facilitate disposition of the PDGF solution in the biocompatible matrix for application at a surgical site, such as a site of fracture in the bone. The kit may also contain instructions for use.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### Preparation of a Composition Comprising a Solution of PDGF and a Biocompatible Matrix

A composition comprising a solution of PDGF and a biocompatible matrix was prepared according to the following procedure.

A pre-weighed block of biocompatible matrix comprising βTCP and collagen was obtained. The β-TCP comprised pure β-TCP particles having sizes ranging from about 100 μm to about 300 μm. The β-TCP particles were formulated with about 20% weight percent soluble Type 1 bovine collagen binder. Such a β-TCP/collagen biocompatible matrix can be commercially obtained from Kensey Nash (Exton, Pa.).

A solution comprising rhPDGF-BB was obtained. rhPDGF-BB is commercially available from Novartis Corporation at a stock concentration of 10 mg/ml (i.e., Lot # QA2217) in a sodium acetate buffer. The rhPDGF-BB is produced in a yeast expression system by Novartis Corporation (Chiron) and is derived from the same production facility as the rhPDGF-BB that is utilized in the products REGRANEX®, (Johnson & Johnson) and GEM 21S (BioMimetic Therapeutics) which has been approved for human use by the United States Food and Drug Administration. This rhPDGF-BB is also approved for human use in the European Union and Canada. The rhPDGF-BB solution was diluted to 0.3 mg/ml in the sodium acetate buffer. The rhPDGF-BB solution can be diluted to any desired concentration according to embodiments of the present invention.

A ratio of about 3 ml of rhPDGF-BB solution to about 100 g dry weight of the β-TCP/collagen biocompatible matrix was used to produce the composition. The rhPDGF-BB solution was expelled on the biocompatible matrix with a syringe, and the resulting composition was blended and molded in preparation for application at a site of osteonecrosis or a site vulnerable to osteonecrosis.

### Example 2

### Treatment for Osteonecrosis of the Jaw

The method of practice for the biocompatible matrix, for example a bone allograft amended with PDGF would follow traditional dental practices to treat an area of exposed bone. In cases where patients present with ONJ, the following criteria of patient signs and symptoms are evaluated:
1) radiographic evidence of widening of the periodontal ligament; moth-eaten poorly defined radiolucency with or without radio-opaque sequestra;
2) cultures of exposed bone may identify Actinomyces species;
3) clinical evidence of localized absence of mucosal tissue with exposed and necrotic bone;
4) pain may or may not be a symptom.

Following identification of the signs and symptoms noted, the method of practice for the allograft supplemented with PDGF includes the following steps: The dosages of PDGF that may be added to the allograft are described previously in the application and include, but are not limited to the following disclosure. The PDGF solution applied to the allograft may be in a concentration as described above, provided the final amount is sufficient to be clinically effective. PDGF is present in the solution in a concentration ranging from about 0.01 mg/ml to about 10 mg/ml, from about 0.05 mg/ml to about 5 mg/ml, or from about 0.1 mg/ml to about 1.0 mg/ml. PDGF may be present in the solution at any concentration within these stated ranges. In other embodiments, PDGF is present in the solution at any one of the following concentrations: about 0.05 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; or about 1.0 mg/ml. It is to be understood that these concentrations are simply examples of particular embodiments, and that the concentration of PDGF may be within any of the concentration ranges stated above.

Various final amounts of PDGF may be used in the allografts of the present invention, provided the amount is clinically effective. Amounts of PDGF that could be used include amounts in the following ranges: about 1 ug to about 50 mg; about 10 ug to about 25 mg; about 100 ug to about 10 mg; and, about 250 ug to about 5 mg.

A sufficient amount of the allograft containing the PDGF is administered to fill the bone defect after debridement. This volume will differ based on the extent of the sequestrectomy and debridement of the affected bone. The procedure is described in the following sentences. Local anesthesia is administered to anesthetize the affected ONJ-site. A soft-tissue flap is gently raised and reflected from the underlying necrotic bone (i.e., the ONJ-site). The dimensions of the necrotic bone are measured. A conservative sequestrectomy is executed, including removal of cortical bone and extending to marginal bleeding bone and into subjacent alveolar bone. The margins of the debrided cortical bone are measured with calipers and the depth of the removed bone is measured with a periodontal probe. The data are recorded in the patient's chart. The designated dose of rhPDGF-BB (in one embodiment 0.3 mg/mL) is added to a volume of allograft required to fill the resultant defect that will then be placed into the prepared recipient site. The dose of PDGF delivered to the site must be therapeutic. As a consequence of the variability of the bone ‘divots’ (the defect remaining after sequestrectomy and debridement), the amount of PDGF that can be added to a particular divot may vary, provided the amount of PDGF contained in the allograft is a clinically effective amount to treat, prevent or slow the progression of ONJ at the site.

If sufficient mucosal soft tissue is present, a flap, without tension, is prepared to cover the treated ONJ site otherwise the recipient site is permitted to granulate in by secondary intention, which involves granulation tissue filling defects in soft or hard tissues when there may be insufficient ability to close integument or mucosa in the oral cavity.

In the event that sufficient mucosal soft tissue is not present, a resorbable collagen material is placed over the treated site, extending 2-3 mm beyond the osseous margins of the defect, in order to contain the graft material. Sutures are placed in order to maximize graft containment.

The allograft and PDGF may be present in a kit. Specifically, in one embodiment, a kit includes the allograft and a syringe containing a solution of 0.3 mg/ml rhPDGF-BB. The allograft and PDGF solution are mixed in a sterile dappen dish or surgical stainless steel bowl, such that the allograft particles are fully saturated. During this time, the ONJ site is optionally thoroughly debrided using hand instrumentation and final irrigation with sterile saline. The hydrated allograft containing PDGF is then placed to fill the osseous defect, and/or a mucosal soft tissue flap could be prepared as noted above.

### Example 3

### Preparation of a Composition Comprising a Solution of PDGF and a Biocompatible Matrix

In this example, the biocompatible matrix comprises bone allograft, DFDBA, FDBA or DBM. The amount of allograft to be employed in the case of existing osteonecrosis is related to the extent of bone loss. Once the surgeon determines the size of the allograft to be employed, a piece of DFDBA, FDBA or DBM, of desired size, is obtained and the PDGF solution is applied to the allograft in an amount to be effective at the site of application of the allograft.

The PDGF solution applied to the allograft may be in a concentration as described above, provided the final amount is sufficient to be clinically effective. PDGF is present in the solution in a concentration ranging from about 0.01 mg/ml to about 10 mg/ml, from about 0.05 mg/ml to about 5 mg/ml, or from about 0.1 mg/ml to about 1.0 mg/ml. PDGF may be present in the solution at any concentration within these stated ranges. In other embodiments, PDGF is present in the solution at any one of the following concentrations: about 0.05 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; or about 1.0 mg/ml. It is to be understood that these concentrations are simply examples of particular embodiments, and that the concentration of PDGF may be within any of the concentration ranges stated above.

Various final amounts of PDGF may be placed in the allografts of the present invention, provided the amount is clinically effective. Amounts of PDGF that could be used include amounts in the following ranges: about 1 ug to about 50 mg, about 10 ug to about 25 mg, about 100 ug to about 10 mg, and about 250 ug to about 5 mg.

Once the surgical field is exposed for application of the allograft and dead or diseased bone is removed, the PDGF solution is applied to the allograft, and the allograft is inserted into the desired site.

### Example 4

### Prophylactic Treatment for Patients at Risk for ONJ

A patient population at risk for ONJ includes any patient on oral or intravenous bisphosphonates in need of dental surgical treatment, especially procedures considered more invasive or traumatic including but not limited to, dental implant procedures, tooth extractions and periodontal surgery. These patients receive the allograft and PDGF as described in Examples 2 or 3 prophylactically at the treatment sites to prevent the occurrence of ONJ.

### Example 5

### Prophylactic Treatment for Patients at Risk for ORNJ

A patient population at risk for ORNJ includes any patient receiving radiation treatment of the mandible, maxilla or surrounding tissue and bone. These patients receive the allograft containing PDGF prophylactically at the vulnerable sites in the jaw to prevent the occurrence of ORNJ. The composition may be applied by percutaneous injection to a vulnerable site or sites in the jaw.

### Example 6

### Prophylactic Treatment for Patients at Risk for ONJ

A patient population at risk for ONJ includes any patient on oral or intravenous bisphosphonates in need of dental surgical treatment, especially procedures considered more invasive or traumatic including but not limited to, dental implant procedures, tooth extractions and periodontal surgery. These patients receive a solution of PDGF in a pharmaceutically acceptable buffer prophylactically at the treatment sites to prevent the occurrence of ONJ.

### Example 7

### Prophylactic Treatment for Patients at Risk for ORNJ

A patient population at risk for ORNJ includes any patient receiving radiation treatment of the mandible, maxilla or surrounding tissue and bone. These patients receive a solution of PDGF in a pharmaceutically acceptable buffer prophylactically at the vulnerable sites in the jaw to prevent the occurrence of ORNJ. The composition may be applied by percutaneous injection to a vulnerable site or sites in the jaw.

### Example 8

### Treatment for Osteonecrosis of the Jaw

The method of practice application of the composition comprising a solution of PDGF in a pharmaceutically acceptable buffer would follow traditional dental practices to treat an area of exposed bone. In cases where patients present with ONJ, the following criteria of patient signs and symptoms are evaluated:

1) radiographic evidence of widening of the periodontal ligament; moth-eaten poorly defined radiolucency with or without radio-opaque sequestra;
2) cultures of exposed bone may identify Actinomyces species;
3) clinical evidence of localized absence of mucosal tissue with exposed and necrotic bone;
4) pain may or may not be a symptom.

Following identification of the signs and symptoms noted, the method of practice for administration of the composition comprising a solution of PDGF in a pharmaceutically acceptable buffer includes the following steps. The dosages of PDGF are described previously in the application and include, but are not limited to the following disclosure. The PDGF composition may be in a concentration as described above, provided the final amount is sufficient to be clinically effective. PDGF is present in the solution in a concentration ranging from about 0.01 mg/ml to about 10 mg/ml, from about 0.05 mg/ml to about 5 mg/ml, or from about 0.1 mg/ml to about 1.0 mg/ml. PDGF may be present in the solution at any concentration within these stated ranges. In other embodiments, PDGF is present in the solution at any one of the following concentrations: about 0.05 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; or about 1.0 mg/ml. It is to be understood that these concentrations are simply examples of particular embodiments, and that the concentration of PDGF may be within any of the concentration ranges stated above.

Various final amounts of PDGF may be used, provided the amount is clinically effective. Amounts of PDGF that could be used include amounts in the following ranges: about 1 ug to about 50 mg; about 10 ug to about 25 mg; about 100 ug to about 10 mg; and, about 250 ug to about 5 mg.

A clinically effective amount of the composition comprising PDGF is administered to the bone defect after debridement. This amount will differ based on the extent of the sequestrectomy and debridement of the affected bone. The procedure is described in the following sentences. Local anesthesia is administered to anesthetize the affected ONJ-site. A soft-tissue flap is gently raised and reflected from the underlying necrotic bone (i.e., the ONJ-site). The dimensions of the necrotic bone are measured. A conservative sequestrectomy is executed, including removal of cortical bone and extending to marginal bleeding bone and into subjacent alveolar bone. The margins of the debrided cortical bone are measured with calipers and the depth of the removed bone is measured with a periodontal probe. The data are recorded in the patient's chart. The designated dose of rhPDGF-BB (in one embodiment 0.3 mg/mL) is delivered to the site to provide a therapeutic effect. Such delivery may be by syringe application. As a consequence of the variability of the bone ‘divots’ (the defect remaining after sequestrectomy and debridement), the amount of PDGF that can be added to a particular divot may vary, provided the amount of PDGF is a clinically effective amount to treat, prevent or slow the progression of ONJ at the site.

If sufficient mucosal soft tissue is present, a flap, without tension, is prepared to cover the treated ONJ site otherwise the recipient site is permitted to granulate in by secondary intention, which involves granulation tissue filling defects in soft or hard tissues when there may be insufficient ability to close integument or mucosa in the oral cavity.

In the event that sufficient mucosal soft tissue is not present, a resorbable collagen material is placed over the treated site, extending 2-3 mm beyond the osseous margins of the defect, in order to contain the PDGF. Sutures are placed in order to maximize graft containment.

The PDGF may be present in a kit. Specifically, in one embodiment, a kit includes the a syringe containing a solution of 0.3 mg/ml rhPDGF-BB. In another embodiment, a first container comprises PDGF and a second container comprises a pharmaceutically acceptable buffer. The PDGF and buffer are mixed, optionally in the first container, or in a syringe. During this time, the ONJ site is optionally thoroughly debrided using hand instrumentation and final irrigation with sterile saline. The PDGF solution is then placed to fill the osseous defect, and/or a mucosal soft tissue flap could be prepared as noted above.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

1. Use of composition comprising a solution of platelet derived growth factor (PDGF) disposed in a biocompatible matrix in the preparation of a medicament for treating, preventing or slowing the progression of osteonecrosis of a jaw or osteoradionecrosis of a jaw in a patient.

2-15. (canceled)
